(19) United States
(12) Patent Application Publication
(10) Pub. No.: US 2007/0135504 A1
(43) Pub. Date: Jun. 14, 2007

(54) Method of Treating and/or Preventing Inflammatory Diseases, including many Autoimmune and Neurological diseases, using drugs to modulate the VDR, and/or PPAR, and/or GCR and/or CB1 nuclear and GPCR receptors; in conjunction with antibiotics which target prokaryotic protein translation

(76) Inventor: Trevor Gordon Marshall, Thousand Oaks, CA (US)

Correspondence Address: TREVOR GORDON MARSHALL, 3423 HILL CANYON AVENUE, THOUSAND OAKS, CA, 91360-1122, US

(21) Appl. No.: 11/608,838

(22) Filed: Dec. 10, 2006

Related U.S. Application Data
(60) Provisional application No. 60/597,574, filed on Dec. 11, 2005.

(51) Int. Cl. A61K 31/4184

(52) U.S. Cl. 514/381; 514/394

(57) ABSTRACT

This invention discloses a method of killing the stealthy intra-cellular bacteria which are key to the pathogenesis of Th1 immune conditions including Diabetes Type 1, Diabetes Type 2, Rheumatic Arthritis, Reactive Arthritis, Osteo Arthritis, Psoriasis, Scleroderma, Osteoporosis, Atherosclerosis, Myocarditis, Endocarditis, Pericarditis, Stroke, Embolism, Alzheimer's, Cystic Fibrosis, Hashimoto's Thyroiditis, Graves Disease, Leprosy, Syphilis, Lyme, Chronic Lyme, Borreliosis, Neuro-borreliosis, Inflammatory Bowel Disease (IBD), Tuberculosis, Latent Tuberculosis, Sarcoidosis, Neurosarcoidosis, Lupus, Discoid Lupus, Lupus Pernio, Lupus Nephritis, Systemic Lupus Erythematosis (SLE), Asthma, Macular Degeneration, Uveitis, Crohn's, Irritable Bowel Syndrome, Sjogren's, Fibromyalgia, Chronic Fatigue Syndrom (CFS), Chronic Fatigue Immune Dysfunction Syndrome (CFIDS), Myalgic Encephalitis (ME), Amyotrophic Lateral Sclerosis (ALS), Parkinson's, Multiple Sclerosis, Autism Spectrum Disorder (ASD), Attention Deficit Disorder (ADD), Attention Deficit Hyperactivity Disorder (ADHD), Schizophrenia, Obsessive Compulsive Disorder (OCD), Dysthymia, Bipolar Disorders, Epilepsy, Dementia, and Mania. This invention achieves its objective partly by reducing the ability of these tiny L-form, intra-phagocytic bacteria to translate proteins within their 70S Ribosome. The 30S and 50S subunits of the bacterial ribosome are targeted both individually, and collectively, with symbiotic bacteriostatic antibiotics. This invention also discloses how to restore the competence of the VDR Nuclear Receptor, key to the innate immune system, by using VDR agonist(s). It additionally discloses how to modulate the availability of endogenous ligands to the PPAR, GCR and CB1 receptors, conditioning the innate immune system to more easily recognize and kill these tiny bacterial pathogens.

This application claims benefit of priority from U.S. Provisional Patent Application Ser. No. 60/597,574 filed Dec. 11, 2005, “A method to use Angiotensin Receptor Blockers, Statins, and functionally similar ligands, to block VDR, PPAR and GPCR in human and/or bacterial genomes, in order to treat and/or prevent infectious diseases, including MRSA and Tuberculosis, Th1 immune and Autoimmune Diseases, Cancers, and HIV/AIDS”; which is incorporated herein by reference in its entirety for any purpose.

Certain aspects of this application claim the benefit of priority from U.S. patent application Ser. No. 11/161,318 (2005) “Treatment and Prevention of Th7 and Autoimmune Diseases effected with Antibiotics and/or Angiotensin Inhibition” and U.S. provisional patent application 60/522,007 (2004) “Treatment and Prevention of Th1 and ‘Autoimmune’ Diseases effected with Antibiotics and/or Angiotensin Inhibition.”

### FIELD OF THE INVENTION

The present invention relates to a method of treating and/or preventing inflammatory diseases, including many ‘autoimmune’ and neurological diseases. This invention differs materially from prior-art in several respects, most notably as it discloses that these diseases are the result of activity of tiny, intra-phagocytic prokaryotic pathogens, and discloses a method for killing these pathogens, the root cause of the disease morbidity, thereby inducing patient recovery, and/or preventing the occurrence and/or preventing the re-occurrence, of these diseases.

Definition of Terms

The term “treat”, “treating”, or “treatment,” in all grammatical forms, as used herein refers to the prevention, reduction, or amelioration, partial or complete alleviation, or cure of a disease, disorder, or condition, wherein prevention indicates treatment of a person at risk for developing such a disease, disorder or condition.

VDR is the commonly accepted acronym for the “Vitamin D Receptor,” a type-1 nuclear receptor, active in the nucleus of many types of cells, especially phagocytes. PPAR is the “Peroxisome Proliferator Activated Receptor,” another type-1 nuclear receptor, and is commonly found in subtypes alpha and gamma. GCR is the “Glucocorticoid Receptor,” again a type-1 nuclear receptor.

The CB1 receptor is a G-Protein Coupled Receptor (GPCR) commonly known as a “Cannabinoid Receptor,” expressed on the surface of Lymphocytes, and active in the immune system. AG2R is also a GPCR, the Angiotensin II Type 1 receptor, active in the Renin-Angiotensin System (RAS).

For the purpose of this patent and Claims, all Prokaryotic organisms shall hereinafter be termed ‘bacteria’, but such term shall also include all archaeal and protozoal pathogens whose genomes carry the code to create proteins functionally similar and/or complementary to G-Coupled Protein Receptors and Nuclear Receptors. For example, but without limitation, the archeal species ‘Archaeoglobus fulgidus’ contains GPCR proteins, including Swiss-Prot:028474.

For the purpose of this specification and claims, the terms ‘Th1 Disease’ and “Th1 Immune Disease” will be used to denote a disease characterized by inflammation involving the presence of the cytokine Interferon-gamma, and/or the presence of intra-phagocytic bacterial pathogens, and/or the presence of a dysregulated Vitamin-D metabolism. The following diseases are examples of Th1 immune diseases: Diabetes Type 1, Diabetes Type 2, Rheumatic Arthritis, Reactive Arthritis, Osteo Arthritis, Psoriasis, Scleroderma, Osteoporosis, Atherosclerosis, Myocarditis, Endocarditis, Pericarditis, Stroke, Embolism, Alzheimer's, Cystic Fibrosis, Hashimoto's Thyroiditis, Graves Disease, Leprosy, Syphilis, Lyme, Chronic Lyme, Borreliosis, Neuro-borreliosis, Inflammatory Bowel Disease (IBD), Tuberculosis, Latent Tuberculosis, Sarcoidosis, Neurosarcoidosis, Lupus, Discoid Lupus, Lupus Pernio, Lupus Nephritis, Systemic Lupus Erythematosis (SLE), Asthma, Macular Degeneration, Uveitis, Crohn's, Irritable Bowel Syndrome, Sjogren's, Fibromyalgia, Chronic Fatigue Syndrom (CFS), Chronic Fatigue Immune Dysfunction Syndrome (CFIDS), Myalgic Encephalitis (ME), Amyotrophic Lateral Sclerosis (ALS), Parkinson's, Multiple Sclerosis, Autism Spectrum Disorder (ASD), Attention Deficit Disorder (ADD), Attention Deficit Hyperactivity Disorder (ADHD), Schizophrenia, Obsessive Compulsive Disorder (OCD), Dysthymia, Bipolar Disorders, Epilepsy, Dementia, and Mania.

Similarly, the term “Th1 Inflammation” will be used to denote inflammation associated with the diagnoses cited in the preceding paragraph as “Th1 Diseases.”

### BACKGROUND OF THE INVENTION

It is currently believed that the diseases known as “autoimmune” diseases are caused by the body's immune system attacking the body itself. This application discloses that many of the ‘autoimmune’ diseases actually form a subset of the class of inflammatory disease characterized by a Th1 cytokine profile, with paracrine Interferon-gamma, the cytokine characteristic of Th1 inflammation, and are also associated with an over-abundance of the secosteroid hormone 1,25-dihydroxyvitamin-D (1,25-D) in the inflamed body tissue. This hormone; 1,25-D; is both a paracrine cytokine and a seco-steroid hormone. Further, this application discloses that the Th1 diseases are caused by multiple species of very tiny L-form Cell-Wall-Deficient (CWD) antibiotic-resistant bacteria living within the cytoplasm of cells, including the phagocytic cells (monocytes, macrophages, lymphocytes, neutrophils and polymorphonuclear cells) (‘leukocytes’) of the immune system itself. Further, this application discloses that killing these stealthy bacteria stops the progression, can prevent re-occurrence, and can even prevent the initial occurrence, of these inflammatory diseases.

It is currently believed that neurological conditions have no pathogenic causation. This application discloses that many neurological diseases actually form a subset of the Th1 class of inflammatory disease involving the presence of the cytokine Interferon-gamma, and/or the presence of intra-phagocytic bacterial pathogens, and/or the presence of a dysregulated Vitamin-D metabolism. Further, this application discloses that the Th1 diseases are caused by multiple species of very tiny L-form Cell-Wall-Deficient (CWD) antibiotic-resistant bacteria living within the cytoplasm of cells, including the phagocytic cells (monocytes, macrophages, lymphocytes, neutrophils and polymorphonuclear cells) (‘leukocytes’) of the immune system itself. Further, this application discloses that killing these stealthy bacteria stops the progression, can prevent re-occurrence, and can even prevent the initial occurrence, of these neurological conditions.

Activation and Re-Activation of the Immune System

The tiny intra-phagocytic L-form bacteria which cause these diseases were first described at Lister Institute in 1934. The “L” of “L-form” denotes the excellent work done at the Lister Institute in isolating them. However, L-forms were never identified as being pathogenic, or as the cause of disease. For example, although an excellent description is given in: KLIENEBERGER-NOBEL E. Filterable forms of bacteria. Bacteriol Rev. 1951 June; 15(2):77-103. Available at URL http://www.pubmedcentral.nih.gov/articlerender.fcgi?artid=440979; during the ensuing half-century these pathogens have not been previously identified as the cause of Th1 immune disease.

This inventor is the first to have described (in detail) the molecular mechanisms by which such organisms are indeed pathogenic, and has devised both molecular biology and clinical trials which demonstrate that they are at the heart of much chronic disease (see the this inventor's published scientific papers in “Special Considerations for Asserted Therapeutic or Pharmacological Utilities” below).

The immune system is activated by a number of mechanisms in the body. One of the most important is activation of the Type-1 Nuclear Receptors in the nucleus of phagocytic cells. This activation commences the process of transcription of DNA genes into the proteins and peptides which drive both the adaptive and the innate immune responses.

The ‘Vitamin D Receptor’ (VDR) has many biological functions in the body, acting on hormone systems as disparate, and ubiquitous as the Para-Thyroid Hormone (PTH) and the Gonadotrophic Releasing Hormone (GnRH). It is principally activated by the seco-steroid hormone called 1,25-dihydroxyvitamin-D (1,25-D).

The VDR has a primary role to play in activation of innate immunity, as it effects, inter alia, expression of Toll-Like Receptors TLR2 and TLR4. These are essential for recognition of the lipoproteins and lipopolysaccharides by which the immune system senses bacteria. The VDR is also responsible for expression of the Cathelicidin Anti-Microbial Peptides, and beta-Defensin Anti-Microbial-Peptides, the body's endogenous ‘antibiotics’.

A biochemical or chemical compound (a ‘ligand’) can bind into the ligand binding domain (LBD) of the VDR and activate the receptor. Alternatively it might deactivate the receptor by binding into a position which restricts the activation by its natural hormone; 1,25-D. Its behavior is determined by whether the inter-atomic forces allow the nuclear receptors to bind with their correct promoter molecules, or not. Both activation and deactivation (agonism and antagonism) are described in this inventor's paper: Marshall T G, Lee R E, Marshall F E: Common angiotensin receptor blockers may directly modulate the immune system via VDR, PPAR and CCR2b. Theor Biol Med Model. 2006 Jan. 10; 3(1):1. Available from URL http://www.tbiomed.com/content/3/1/1

In particular, an exogenous chemical agent can activate or deactivate VDR in this manner (by functioning as a ligand). Such a ligand can also bind into the Peroxisome Proliferator-Activated Receptors (PPAR) and the Glucocorticoid Receptor (GCR). Common ‘Sartans’, and some ‘Statins’, already approved by the US FDA for moderate hypertension and hyperlipidemia, are just a few of the compounds which can bind into these receptors

Such activation and de-activation conditions the operation of the immune system, and is effective in therapy and/or prevention of Th1 inflammatory disease. This inventor has discovered, and disclosed, how to quantify the degree of such agonistic and antagonistic activity. Table 1 shows the molecular affinities of the common classes of drugs called Sartans and Statins, calculated for several Nuclear Receptors active in the immune system. A subset of this table was recently published in The Lancet, Marshall T G: Are statins analogs of vitamin D? Correspondence to Grimes, D S. The Lancet 2006; 368:1234.

Note that Table 1 shows considerable differences in immune receptor activity between the various Sartans and Statins currently licensed by the FDA as safe for therapeutic use in their primary indications. It is clear that no direct equivalence can be drawn between the members in each family of drugs. Further, it is not obvious from Table 1 whether a drug is agonistic or antagonistic to receptor activation, and considerable theoretical and geometric analysis is required to deduce this. However, this inventor has recently reported that Simvastatin is a partial agonist of the VDR—see, for example: Marshall T G. “Are Statins Analogues of Vitamin D?” Correspondence to Grimes, D S. The Lancet 2006, 368:1234.

However, even though Simvastatin is a partial VDR agonist it is not as effective as the sartan Olmesartan Medoxomil in treatment and/or prevention of Th1 inflammation, as it does not affect other immune-system receptors in the same beneficial manner as Olmesartan (see, for example, Photograph 6, Photograph 11 and Photograph 12).

The right-hand panel of Photograph 12 shows both Olmesartan and 1,25-D in the configurations they dock into the VDR, allowing easy evaluation of the agonistic activity of Olmesartan, which is primarily conditioned on C14, O1, O2 and the corresponding hydroxyl on C15, which together stabilize ARG274, TYR143, SER237 and SER275, the amino acid residues of the VDR which enable activation of the VDR. Detailed analysis of the inter-atomic force interactions is shown in Photograph 11.

The left hand panels of Photograph 12 show the configurations of the key Vitamin-D metabolites when they dock into the VDR. It can be seen that only 1,25-D has the 1-alpha-hydroxylation needed to stabilize ARG274, SER237 and SER 275. The remaining Vitamin-D metabolites, principally Vitamin-D, 25-hydroxyvitamin-D, 24,25-dihydroxyvitamin-D and 25,26-dihydroxyvitamin-D act as VDR antagonists, displacing the active hormone 1,25-D from the VDR in a concentration and affinity dependent manner. Exogenous Vitamin-D is thus immunosuppressive, and must be minimized during any therapy to treat and/or prevent Th1 inflammatory conditions.

Additionally, Olmesartan Medoxomil is a partial agonist of the CB1 receptor (see Photograph 13).

Even though Simvastatin does not activate the CB1, it does partly activate the VDR, and is a ligand for PPAR, thus beneficially affecting innate immunity (see Photograph 9)

Weakening the Pathogens

Such chemical compounds (ligands) can independently, and/or in addition, bind into the ligand binding domain of the G-Protein Coupled Receptors (GPCRs) located in the genomes of pathogens, including bacterial, archaeal and protozoal pathogens, including the pathogens which cause morbidity in Th1 immune dysfunction. When such prokaryotic GPCRs are blocked by ligands, the pathogenic organism cannot function correctly, and the pathogen eventually dies. Thus the blockade of the pathogen's GPCRs and NRs gives such a ligand antimicrobial and/or anti-bacterial and/or antibiotic properties. Common Sartans, and some ‘Statins,’ already approved by the FDA for moderate hypertension and hyperlipidemia, are just a few of the compounds which can bind into the pathogens' GPCRs and NRs. By careful selection of the Statin, Sartan, or functionally-equivalent ligand, to be used in prevention and/or treatment of Th1 inflammatory disease, a compound can be selected which additionally have action directly against the pathogenic genomes.

Examples of such direct action of these drugs on Pathogenic genomes are shown in Photo 1 (Olmesartan Medoxomil in MRSA SAR0276), Photo 2 (Olmesartan Medoxomil in bb0006 from Borrelia burgdorferi), Photo 3 (Olmesartan Medoxomil in rp630 from species Rickettsia), photo 4 (Olmesartan Medoxomil in E. coli ydgG), Photo 5, Olmesartan Medoxomil in Mycobacterium tuberculosis mt1133), and Photo 7 (Simvastatin in MRSA SAR0276).

This invention additionally discloses a method to kill the stealthy L-form bacteria by reducing their ability to transcribe DNA genes into proteins with their 70S Ribosome. The 30S and 50S subunits of the bacterial ribosome are targeted both individually and collectively by the antibacterial agents optionally used as part of this invention. Further, this invention reduces the availability of Angiotensin II to the Angiotensin receptors on the phagocytic outer membrane, which decreases the amount of Nuclear-Factor-kappa-B produced to fuel the transcription of Cytokines and Chemokines by the activated phagocyte.

Optimally Effective Antibiotics and Dosing Regimes

These stealthy intra-cellular bacteria are so very small because they have shed parts of their physical structure (their Cell Walls), and often also some of their plasmids, in the transformation into the L-form pathogens. Therefore, analysis of the actions of antibiotics on these organisms can only be performed after analysis of the bacterial genome, just as was performed to obtain the mathematical structural protein analyses shown in Photographs 1,2,3,4,5, and 7.

The postulates of Koch do not apply to L-forms in chronic disease, as horizontal transfer of DNA between chromosomes, and exchange of plasmids, become dominant; making identification of any one definitive organism a fruitless endeavor.

These L-form bacteria are very, very, difficult to culture in-vitro, and conventional antibiotic sensitivity testing offers little or no help in understanding the effectiveness of this invention. However, this inventor has demonstrated how the intra and extra-cellular behavior of these bacteria can be revealed with ‘phase-contrast’ optical microscopy.

Standard antibiotic regimes do not kill these intra-phagocytic bacteria. They may therefore be thought of as “antibiotic-resistant” bacteria. In their L-forms, they are not susceptible to antibiotics in most common use, the ‘bactericidal’ antibiotics. See, for example:
Dienes L, et al: The Transformation of Typhoid Bacilli into L Forms under Various Conditions. J. Bacteriol. 1950 June; 59(6): 755-764. PMID:15436450
Dienes L: The Isolation of L Type Cultures from Bacteroides with the Aid of Penicillin and Their Reversion into the Usual Bacilli. J Bacteriol. 1948 October; 56(4): 445-456. PMID:16561593

This invention can kill these antibiotic-resistant bacteria. One problem is that as the bacteria are killed they release endotoxins, and/or other toxic biochemicals, into the cytoplasm, causing further disease symptoms, sometimes of even higher intensity than during the usual activity of the Th1 disease itself. This can be likened to the Jarisch-Herxheimer Reaction which has been documented when killing acute bacterial pathogens, most notably when killing the Treponema pallidum which are believed to cause Syphilis. A more descriptive term has recently become accepted to describe this malaise—“Immunopathology.”

This invention solves the problem of Immunopathology—including induced anaphylaxis by targeting the bacterial genome—carefully controlling the pathogen's environment, as well as the antibiotic selection and dosing regimens.

Even if the patient is given a conventional dosage of the same antibiotics described in this invention, the antibiotics may fail to totally kill the pathogens, and if they do, there is the risk of a cytokine release sufficiently intense to cause life-threatening cardiac bradycardia or life-threatening pulmonary insufficiency, both of which were observed during experimentation with this invention.

This inventor has previously performed research on novel drug-dosing regimes in several diseases. In 1982, when studying Diabetes, this inventor pioneered increased efficacy and reduced side-effects from a continuous infusion of Insulin, and in Cryptorchidism and Infertility he explored increased efficacy of pulsatile dosing of the hormones LH-RH and Gn-RH.

One of the best ways to administer a continuous concentration of any drug is by using an infusion pump, like the transcutaneous infusion pump for Insulin he invented in 1982: Marshall T G, Mekhiel N, Jackman W S, Perlman K, Albisser A M. New microprocessor-based insulin controller. IEEE Trans Biomed Eng. 1983 November; 30(11):689-95.

During his Doctoral Research he also explored pulsatile administration of drugs, and his research group was able to cure Cryptorchidism and Infertility by using pulsatile injections of hormone, rather than using a continuous concentration: Keogh E J, MacKellar A, Mallal S A, Dunn A G, McColm S C, Somerville C P, Glatthaar C, Marshall T. Attikiouzel J: Treatment of cryptorchidism with pulsatile luteinizing hormone-releasing hormone (LH-RH). J Pediatr Surg. 1983 June; 18(3):282-3.

It is typically believed that antibiotics administered at doses below the Minimum Inhibitory Concentration (MIC) are ineffective, and are likely to encourage the formation of antibiotic-resistant forms of the bacteria. However, this application discloses that in order to kill the antibiotic-resistant intra-cellular bacterial L-forms which cause Th1 inflammation, antibiotics blocking bacterial protein synthesis by inhibiting the function of the 70S bacterial Ribosome are needed. Moreover they are often optimally effective when delivered in a pulsatile fashion, wherein the peak concentration in the bloodstream may or may not be in excess of the MIC, but where the antibiotic concentration is allowed to decay away to a lower value before the next dose of antibiotic is given.

It should be noted that the simplified pharmacokinetic model, which is usually used to describe antibiotic absorption, anticipates an exponential rise of concentration to the peak value, and then a single exponential decay of that concentration (which is considered to be distributed within the plasma compartment). A pseudo-continuous concentration in the bloodstream can be achieved by dosing the drug at sufficient frequency that the next dose is absorbed before significant exponential decay from the previous dose.

The 70S Bacterial Ribosome

These L-form bacteria synthesize proteins (which they need for their survival) within a structure called a Ribosome. The bacterial ribosome is termed a ‘70S Ribosome’ and it is conventionally divided into two subunits called the ‘30S’ and the ‘50S’ subunits.

The function of the 30S subunit is primarily determined by the 16S RNA of which it is primarily comprised, while the 50S subunit's function is primarily determined by the bacterial 23S RNA. Both subunit structures are completed by a variety of proteins and additional smaller RNA elements.

Antibiotics which inhibit the 30S subunit typically bind in the region near the helix which ‘advances’ during the transcription of bacterial mRNA to bacterial proteins. Antibiotics which inhibit the 50S subunit typically bind in the region where the tRNA docks, or in the region termed the Peptidyl Transferase Center (PTC), or in the region (the ‘tunnel’) where the partially assembled protein travels through the body of the 50S subunit, prior to emerging as a completed protein from the ribosome.

Antibiotics which act by inhibiting actions of the 30S ribosomal subunit include Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amakacin, Netilmicin, Neomycin,

Antibiotics which act by inhibiting actions of the 50S ribosomal subunit include Azithromycin, Clarithromycin, Clindamycin, Chloramphenicol, Linezolid, Erythromycin, Roxithromycin, Troleandomycin, Tylocin, Sparsomycin, Carbomycin A, Sparsomycin, Lincomycin, Cethromycin, Telithromycin, Tiamulin, Dalfopristin and Quinupristin.

The effectiveness of this invention is partially due to its restoration of homeostasis in the body's innate immune system, and partially due to the use of antibiotics which act symbiotically on different areas of the ribosome, reducing the statistical likelihood that any bacteria species will have developed resistance mechanisms which simultaneously overcome all the methods being used by this invention to weaken protein synthesis by the 70S ribosome.

The Vitamin D Metabolites in Th1 Inflammation.

Th1 inflammation is customarily defined as inflammation which produces an inflammatory cytokine profile including significant ‘Interferon-gamma.’ Moreover, since this Th1 cytokine release also increases the concentration of the secosteroid hormone 1,25-dihydroxyvitamin-D (1,25-D) in the infected phagocytes, it is often possible to measure the proportion of 1,25-D which leaches into the bloodstream, together with plasma 25-hydroxyvitamin-D (25-D), and estimate the extent of Th1 process in well-perfused, inflamed tissue.

One estimate of Th1 inflammation is performed by calculating the D-Ratio, the ratio of 1,25-D (in pg/ml) to the 25-D (in ng/ml). The value for a healthy population is 1.25, and this ratio is often elevated in Th1 immune disease because 25-D is down-regulated, and energetically converted to 1,25-D in the cytoplasm of the phagocytes and leukocytes. The conversion of 25-D to 1,25-D is catalyzed by the action of the cytokine Interferon-gamma. The measurement and prediction based on serum-based assays of the D-metabolites is only valid if the patient is not taking any supplements containing Vitamin D, and where the value of the presenting 25-D assay is between 12 ng/ml and 20 ng/ml.

This inventor has discovered that levels of 25-D higher than 25 ng/ml are associated with that metabolite exerting immunosuppression. 25-D reduces innate immune activity by displacing 1,25-D from the ligand binding domain of the VDR and de-activating that receptor, preventing the transcription of genes essential to proper functioning of the innate immune system.

Many investigators have noted that the level of 25-D falls below 20 ng/ml in patients with the Th1 inflammatory diseases, but that observation has not heretofore been recognized as a useful marker for the disease process itself. It has been mistakenly linked with an aberrant calcium metabolism. The calcium metabolism is, however, primarily regulated by the Para Thyroid Hormone (PTH) and the calcium-sensing receptor (CASR). See, for example: Thakker R V: Disorders of the calcium-sensing receptor. Biochim Biophys Acta. 1998 Dec. 10; 1448(2):166-70.

Further data and information about the behavior of the D metabolites in Th1 inflammation can be found in this inventor's collaborative publication: Waterhouse J C, Marshall T G, Fenter B, Mangin M, Blaney G: High levels of active 1,25-dihydroxyvitamin D despite low levels of the 25-hydroxyvitamin D precursor—Implications of dysregulated vitamin D for diagnosis and treatment of Chronic Disease. In Vitamin D: New Research. Volume 1. Edited by: Stoltz V D. New York. Nova Science Publishers, 2006. ISBN: 1-60021-000-7

Optimally Effective Dosing Regimes for Ligands Acting on the GPCR and NR

The subject compounds/ligands exhibit different affinities for different receptors. As the bloodstream concentration of each ligand is changed, they will have different effects on different receptors. An example chart showing such sensitivity variation for the ARBs is given in our paper “Marshall T G, Lee R E, Marshall F E: Common Angiotensin Receptor Blockers may directly modulate the immune system via VDR, PPAR and CCR2b. Theoretical Biology and Medical Modelling, 2006 Jan. 10; 3(1):1.

Consequently the choice of ligands is important. One must employ ligands with good affinity for the receptors one wants to block, a good affinity for those one wants to agonize, and with minimum affinity for those receptors which are necessary for proper functioning of the rest of the body.

Additionally, the concentration of ligand in the bloodstream should be kept relatively constant, so as to minimize the health of the pathogen(s). Too high a peak concentration may affect receptors which are not being targeted.

This inventor has previously performed research on novel drug-dosing regimes in several diseases. In Diabetes, he explored increased efficacy and reduced side-effects from a continuous infusion of Insulin, and in Cryptorchidism and Infertility he explored increased efficacy of pulsatile dosing of the hormones LH-RH and Gn-RH.

During his Doctoral Research he also explored pulsatile administration of drugs, and his research group was able to cure Cryptorchidism and Infertility by using pulsatile injections of hormone, rather than using a continuous concentration: Keogh E J, MacKellar A, Mallal S A, Dunn A G, McColm S C, Somerville C P, Glatthaar C, Marshall T, Attikiouzel J: Treatment of cryptorchidism with pulsatile luteinizing hormone-releasing hormone (LH-RH). J Pediatr Surg. 1983 June; 18(3):282-3.

One of the best ways to administer a continuous concentration of any drug is by using an infusion pump, for example the transcutaneous infusion pump for Insulin I invented in 1982: Marshall T G, Mekhiel N, Jackman W S, Perlman K, Albisser A M: New microprocessor-based insulin controller. IEEE Trans Biomed Eng. 1983 November; 30(11):689-95.

Other methods of administering semi-continuous concentrations of any drug are transcutaneous patches, sub-dermal implanted ‘reservoirs’, controlled-release drug compounding formulations, controlled-release binders (such as polymers) and implanted infusion pumps.

For example, the administration of Statins and Sartans at intervals beyond about 8 hours causes them to lose efficacy, because the concentration in the blood stream drops below the level at which they begin to be displaced from the receptors by endogenous (undesirable) ligands (e.g., cytokines). Thus, the conventional dosing of the ARBs that the FDA has approved for hypertension, 24 hourly, with “the amount of return on twice daily dosing . . . already a poor investment,” does not allow them to function effectively as pathogenic antagonists. They must be dosed much more frequently (preferably semi-continuously) so as to apply the maximum possible desirable effects upon the receptors in the inflamed tissue.

Immunopathology

It is not possible to kill the intra-phagocytic pathogens without significant malaise being caused to the patient by immunopathology. Immunopathology is the term given to the way in which the immune system affects the rest of its body while the immune system is doing its job, killing the pathogens.

As intra-cellular pathogens are killed, many of the cells containing those pathogens will undergo apoptosis, or even suffer physical destruction. The body will lose both white and red cells during the course of the healing process. The key to an effective therapy is therefore to control the degree of bacterial killing to a level which the patient's body can handle without significant additional suffering or damage. This prolongs the period needed until the end-point of recovery, with complete recovery typically taking 6 to 48 months, depending on the diagnosis, and the bacterial load.

The invention disclosed herein achieves this goal by using very low doses of antibiotics, and therefore it allows the immune system, once it is activated by the ligands herein disclosed, to be the primary killer of the pathogens. Further, the antibiotics are targeted at the 70S Bacterial Ribosome, which allows a concentration-dependant efficacy. One molecule of antibiotic binds into one ribosome, weakening one bacterium. This ensure that the resulting cell-death, and associated cytokine storm, occurs at a controlled rate.

Special Considerations for Asserted Therapeutic or Pharmacological Utilities

With respect to MPEP 2107.03 “Special Considerations for Asserted Therapeutic or Pharmacological Utilities,” the utility of this methods patent has been established by “statistically relevant data documenting the activity of a compound or composition, arguments or reasoning, documentary evidence (e.g., articles in scientific journals), or any combination thereof.”

In addition to the science disclosed in this patent, a sampling of peer-reviewed papers and conference presentations (from this inventor) sufficient to establish such utility, which also document “actual evidence of success in treating humans” during the Phase 2 clinical studies conducted by this inventor, includes:
Marshall T G: Are statins analogs of vitamin D?. Correspondence to Grimes, D S. The Lancet 2006; 368:1234 doi:10.1016/S0140-6736(06)69509-3
Marshall T G: A New Approach to Treating Intraphagocytic CWD Bacterial Pathogens in Sarcoidosis, CFS, Lyme and other Inflammatory Diseases. American Academy of Environmental Medicine; 2006, Plenary Sessions Syllabus, 41 st Annual Meeting
Marshall T G. What is the role of CWD bacteria during HIV infection? Invited Conference Presentation, ‘Recovering from Chronic Disease’, Jun. 17, 2006, Publisher: Autoimmunity Research Foundation (DVD transcript available)
Marshall T G: VDR Nuclear Receptor Competence is the Key to Recovery from Chronic Inflammatory and Autoimmune Disease. Abstract presentation, Days of molecular medicine, 2006. Copy available from URL http://autoimmunityresearch.org/karolinska-handout.pdf
Marshall T G, Lee R E, Marshall F E: Common Angiotensin Receptor Blockers may directly modulate the immune system via VDR, PPAR and CCR2b, Theoretical Biology and Medical Modelling, 2006 Jan. 10; 3(1):1. Available from URL http://www.tbiomed.com/content/3/1/1
Marshall T G: Molecular genomics offers new insight into the exact mechanism of action of common drugs—ARBs, Statins, and Corticosteroids. FDA CDER Visiting Professor presentation, FDA Biosciences Library, Accession QH447.M27 2006. Copy available from URL http://autoimmunityresearch.org/fda-visiting-professor-7mar06.ram
Marshall T G, Marshall FE: Sarcoidosis succumbs to antibiotics—implications for autoimmune disease. Autoimmunity Reviews, 2004; 3(4):295-3001.
Waterhouse J C, Marshall T G, Fenter B, Mangin M, Blaney G: High levels of active 1,25-dihydroxyvitamin D despite low levels of the 25-hydroxyvitamin D precursor—Implications of dysregulated vitamin D for diagnosis and treatment of Chronic Disease. In Vitamin D: New Research. Volume 1. Edited by: Stoltz V D. New York: Nova Science Publishers; 2006. ISBN: 1-60021-000-7
Marshall T G, Fenter B J, Marshall F E: Antibacterial Therapy Induces Remission in Sarcoidosis (in English). JOIMR 2005; 3(1):2 Available from URL http://www.joimr.org/phorum/read.php?f=2&i=107&t=107
Marshall T G, Fenter B, Marshall F E: Antibacterial Therapy Induces Remission in Sarcoidosis. Herald MKDTS 2004g; Volume III: Release 1. (The Journal of the Interregional Clinical-Diagnostic Center, Kazan, published in Russian translation). Invited Paper. Special issue on Sarcoidosis. ISSN: 1726-6149
Marshall T G, Fenter B, Marshall F E: Putative Antibacterial Mechanisms for Angiotensin Receptor Blockers. JOIMR 2004; 2(2):1.
Marshall T G, Marshall F E: Sarcoidosis succumbs to antibiotics—implications for autoimmune disease. Autoimmunity Reviews, 2004; Supplement 2:55 (Abstracts of 4th International Congress on Autoimmunity)
Marshall T G: Bacterial Th1 Processes Seem Key to Chronic Lyme Remission. ILADS conference, October 2004, Rye Town, N.Y.
Marshall T G: How Borrelia Evades the Immune System, and How we Help it Kill This Th1 Bacterium. ‘30th Anniversary of Lyme Disease’ conference, Farmington, Conn., May 7, 2005
Marshall T G, Mangin M, Marshall F E: Bacterial Th1 Processes Key to CFS/ME Remission. AACFS conference, Madison, Wis., October 2004
Marshall T G: Genomics, Molecular Medicine and Antibiotic Resistance. ‘Recovery From Chronic Disease’ conference, Chicago, Ill., Mar. 12, 2005.
   Disclosure of “Best Method” Implementation

The most effective method to kill the Th1 pathogens is to administer Olmesartan Medoxomil, 40 milligrams (mg) every 6 hours, together with the 30S ribosomal sub-unit inhibitor Minocycline Hydrochloride, 100 mg administered at a frequency of one dose every 48 hours; together with the 50S ribosomal sub-unit inhibitor Azithromycin, 125 mg administered at a frequency of one dose every 10 days; and the symbiotic 50S inhibitor Clindamycin, 150 mg administered at a frequency of one dose every 48 hours.

The initial clinical study conducted by this inventor identified the protocol in the preceding paragraph as optimally efficient at killing the L-form bacterial pathogens. But, due to severe and debilitating Immunopathology, most patients were unable to immediately commence at this optimal dosing, and had to slowly ramp up to this antibiotic dose over a period of twelve, or more, months. Each antibiotic must be sequentially introduced, and increased in dose during the period of therapy (up to the values disclosed herein), in order to limit the immunopathology, so that the patient remains functional.

As the intracellular pathogens were killed, and both white and red blood cells underwent apoptosis, and this needs to be carefully monitored by the supervising Physician.

Consideration of Potential ‘Prior Art’.

Patent applications by this inventor containing potentially related disclosures include U.S. provisional patent application Ser. No. 60/597,574 filed Dec. 11, 2005, “A method to use Angiotensin Receptor Blockers, Statins, and functionally similar ligands, to block VDR, PPAR and GPCR in human and/or bacterial genomes, in order to treat and/or prevent infectious diseases, including MRSA and Tuberculosis, Th1 immune and Autoimmune Diseases, Cancers, and HIV/AIDS”, patent application Ser. No. 11/309,355 (2006), “A method of treating and/or preventing Cancers and AIDS using Sartans and/or statins to modulate VDR, and/or PPAR, and/or GCR, and/or CB1 receptors, in conjunction with certain bacteriostatic antibiotics,” US patent application Ser. No. 11/161,318 (2005) “Treatmentand Prevention of Th1 and Autoimmune Diseases effected with Antibiotics and/or Angiotensin Inhibition,” U.S. provisional patent application 60/595,727 (2005) “Treatmentand Prevention of AIDS and Cancers effected with Antibiotics and/or Angiotensin Inhibition,” U.S. provisional patent application 60/522,007 (2004) “Treatment and Prevention of Th1 and ‘Autoimmune’ Diseases effected with Antibiotics and/or Angiotensin Inhibition,” and U.S. provisional patent application 60/597,071 (2005) “CCR2b Chemokine Receptor Antagonists as Treatment or Prevention for Th1 Immune Diseases, Cancers and HIV/AIDS.”

This invention is based on a thorough and complete elucidation of a mathematical model of the pathogenesis of Chronic Disease, a model which has been confirmed by Molecular Genomics, Molecular Biology, and a rapid-prototyping Phase 2 clinical trial.

This inventor recently delivered a presentation at the Karolinska Institute (home of Team Nobel) establishing this inventor's total scientific leadership in the understanding of the cause of, and the treatment of, chronic disease, including AIDS and Cancers: Marshall T G: VDR Nuclear Receptor Competence is the Key to Recovery from Chronic Inflammatory and Autoimmune Disease. Days of Molecular Medicine, 2006. Copy available from URL http://autoimmunityresearch.org/karolinska-handout.pdf

This scientific leadership has resulted in an invention which stands on its own, where there really is no other previously filed patent application which covers the breadth of background, insight, the methods disclosed in, or the utility of, this patent application.

This inventor has uniquely made (inter alia) the following scientific breakthroughs, which led to the detailed science underpinning this invention, and the consequent elegance and uniqueness of the method hereby disclosed:
Recognition that patients suffering from differing ‘autoimmune’ inflammatory conditions shared many aspects in common with each other, and in common with many chronic idiopathic inflammatory conditions
Recognition that the inflammation in such disorders was of type Th1
Elucidation that Th1 inflammation is driven by intraphagocytic pathogens
Recognition that the conditions thought to be ‘autoimmune’ are actually due to a defect in the innate immune system, and do not result from the antibodies of adaptive immunity.
Understanding that the end-point of effective therapy is complete recovery from the disease state (commonly called ‘cure’)
Understanding that malaise due to apoptosis and immunopathology will necessarily occur as the intra-phagocytic pathogens are killed, and recognition that the intensity of this immunopathology must be controlled in any therapy.
Identification that VDR NR competence is key to allowing the immune system to recognize, and kill, the intraphagocytic pathogens, in addition to the roles of the Angiotensin II Type 1 Receptor, the PPAR and GCR NRs, and the Cannabinoid GPCRs in the disease process.
Whereas modern medicine has focused on reducing symptoms, and palliation of the inflammatory condition (primarily by using immunosuppression), this inventor has been focused on providing an alternate intervention leading to full recovery. This intervention is based on his elucidation of the detailed molecular biology of the inflammatory disease processes, rather than on administering drugs and observing their effects.

Nevertheless, the following US Patent Applications have been identified as those which could be examined as potentially containing material which, at first glance, might seem to portend “prior art.”

United States Patent Application 20060135422, “Use of Angiotensin receptor blockers (ARBs) to treat diseases associated with excess ACE,” Inventor David W Moskowitz, describes a method of using Angiotensin Receptor Blocker Drugs (Sartans) in a number of chronic diseases.

Firstly, David W Moskowitz, MD, has been exposed to this inventor's scholarly and online publications, and therefore the priority dates are critical when evaluating the relevance of Dr. Moskowitz's filings.

This application does not contain prior art because it is deficient in its definition of the diseases for which the treatment is proposed. A number of diseases are named, and it is asserted that they are in some way “associated with excess ACE.” Yet Angiotensin Converting Enzyme (ACE) excesses are primarily associated with the disease Sarcoidosis, which is not mentioned in this application (except in one of the citations). Even in Sarcoidosis, lack of association with serum ACE, via both false positive and false negative, occurs in the majority of diagnoses (partly due to genotype variation), and it would be very difficult for an academic to sustain the argument that the disease process is “associated with excess ACE.” The other diseases specified in the application are even less directly related to “excess ACE.”

Moskowitz also fails to expound the impact of the various ACE genotypes on the disease process, instead focusing on D/D, and repeatedly citing his own hypothesis: Moskowitz D W. Is angiotensin I-converting enzyme a “master” disease gene? Diabetes Technol Ther. 2002; 4(5):683-711. PMID:12458570

Yet the theory advanced in that paper has subsequently been deprecated by a wealth of evidence. See, for example: “No relation was thus found between I/D polymorphism and susceptibility to sarcoidosis” (Alia P, et al: Association between ACE gene I/D polymorphism and clinical presentation and prognosis of sarcoidosis. Scand J Clin Lab Invest. 2005; 65(8):691-7. PMID:16319043)

See also, for example, the review: Sayed-Tabatabaei F A, et al. ACE polymorphisms. Circ Res. 2006 May 12; 98(9):1123-3. PMID:16690893

Dr Moskowitz has not contemplated that the ARB might be functioning as an antibacterial agent, or as an immunomodulatory agent. The patent does not disclose that L-form intra-phagocytic bacteria are the root cause of the claimed diseases.

Further, the few clinical trails which Dr Moskowitz conducted have failed to confirm the utility of the method proposed in Moskowitz's patent application. Application of this methodology, an ARB acting alone, in the majority of diseases mentioned in the application, has failed to confirm the inventor's disclosure of the functionality of his method, or even its utility, particularly in Th1 inflammatory diseases.

20060135422 therefore cannot be considered to be “prior art.”

United States Patent Application 20060111397; Moskowitz, David W.; May 25, 2006; “Methods and compositions for treating diseases associated with excesses in ACE” fails to attain the standards for “prior art” under a similar analysis to that recited for 20060135422 (above). Note particularly the priority dates for each aspect claimed.

United States Patent Application 20050203169; Moskowitz, David W; Sep. 15, 2005; “Methods and compositions for treating diseases associated with excesses in ACE,” fails to attain the standards for “prior art” under a similar analysis to that recited for 20060135422 (above). Note particularly the priority dates for each aspect claimed.

United States Patent Application 20030040509; Moskowitz, David W; Feb. 27, 2003; “Methods and compositions for treating diseases associated with excesses in ACE,” fails to attain the standards for “prior art” under a similar analysis to that recited for 20060135422 (above).

United States Patent Application 20060154975, “Modulators of the glucocorticoid receptor, AP-1, and/or NF-kB activity and use thereof” claims “A method of treating a disease or disorder which is associated with the expression product of a gene whose transcription is stimulated or repressed by glucocorticoid receptors, or a method of treating a disease or disorder associated with AP-1- and/or NF-.kappa.B-induced transcription” in Claim 11.

The problem with contemplating an omnibus patent such as 20060154975 is to distinguish those attributes of the Claims which are not novel or useful, from those which possess both novelty and utility.

In this case, I would paraphrase this patent application to be similar in novelty and utility to one which claims “Prevention of death effected by ingestion of water.” The most prolific GCR modulator was devised in 1954, a corticosteroid called Prednisone, and this drug has been used in all of the disease indications cited in the application. So any novelty in 20060154975 is not obvious to this expert.

The novelty apparently is claimed to come from the particular drug formula and configurations that are claimed as non-steroidal GCR modulators. Again, there is little novelty immediately evident because of the breadth of the claimed chemical description, which covers many drugs already patented and accepted to the pharmacopeia.

A “best method” disclosure is not obviously present in 20060154975

Significantly, that application does not disclose the effect of modulating the GCR. Indeed, since mice which are bred GCR-deleted do not survive gestation, there is very little knowledge available on the exact functions of the GCR.

I have disclosed in Table 1 that there are significant similarities between each of the Type 1 nuclear receptors, and their ligands. There is considerable functional overlap. It is difficult to imagine a GCR modulator which does not profoundly affect other Type 1 Nuclear Receptors. So I would even question the applicant had sufficient grasp of the field in which the invention is claimed to make any assertions about supposed utility.

In any case, 20060154975 does not disclose which of the diseases cited in the application are caused by L-form bacteria, and does not disclose how the modulation of the GCR, as distinct from the other receptors, can be an effective treatment for the claimed diseases.

20060154975 cannot therefore be regarded as portending “prior art.”

Indeed, an exhaustive search of the patent literature has confirmed the novelty of this invention, and confirmed that the acknowledged scientific leadership contained in this inventor's peer-reviewed scientific papers is matched by the utility of the disclosed method.

Photos Showing Aspects of Invention

Please note that the following photographs show a portion of the scientific background of the implementation of this invention. For obvious reasons they do not show every ARB binding to every human and bacterial receptor, nor every statin (or other pharmaceutical drug with similar properties) binding to the subject genomes. They are necessary to demonstrate preferred implementations, which would otherwise be difficult to communicate.

Photograph 1 is taken from the screen of a computer which is loaded with a model of the SAR0276 GPCR from the Methicillin-resistant Staphylococcus aureus (MRSA) ‘superbug’ MRSA252, the GPCR called Swiss-Prot:Q6GK32_STAAR, obtained with the procedures described in this inventor's paper “Common Angiotensin Receptor Blockers may directly modulate the immune system via VDR, PPAR and CCR2b” (attached). The GPCR is shown as a customary helix-sheet diagram rather than by showing the thousands of tiny atoms which comprise its detail. An example Angiotensin Receptor Blocker, ‘Olmesartan,’ is tightly bound (Ki=0.9 nmol) in a de-activating location of this GPCR (olmesartan is the yellow, red and blue structure). The MRSA bacterium containing this GPCR would not be able to function as effectively once the Olmesartan had bound to this GPCR.

Photograph 2 shows Olmesartan bound to a GPCR (Swiss-Prot:BB0006) from the Genome of ‘Borrelia burgdorferi’, the species which causes, inter alia, Lyme Disease. The Olmesartan is binding with high efficiency, Ki=0.7 nanomolar.

Photograph 3 shows Olmesartan bound to a GPCR (Swiss-Prot:RP630) from the species ‘Rickettsia prowazekii,’ another ubiquitous human pathogen. Here the ligand is bound even more tightly, with Ki=0.2 nmol.

Photograph 4 shows Olmesartan bound to a GPCR (ydgG) from the species ‘E-coli’ with Ki=0.3 nmol, clearly stopping it from functioning as the bacterium would wish.

Photograph 5 shows Olmesartan bound to the GPCR protein from Mycobacterium Tuberculosis (Swiss-Prot:MT1133) with Ki=1.7 nmol. Again, the full functioning of this terrible bacterium is clearly inhibited by the ligand.

Photograph 6 shows Olmesartan bound into the Homo sapiens VDR NR, with an affinity Ki=10 nmol. Geometric analysis shows high affinity for the helix containing residues Ser278, Arg 274 and Ser 275, and also for the helices containing Ser237 and Tyr143. It is to be expected that anchoring these helices geometrically in space will activate the VDR, and Olmesartan is therefore a partial VDR Agonist (see Photographs 10,11 for detailed atomic interaction graphs showing the forces exerted between the VDR residues and Olmesartan Medoxomil, and 1,25-D. Photos 10 and 11 allow more detailed analysis of the Agonistic action of Olmesartan Medoxomil and the VDR).

Photograph 7 shows the Statin ‘Simvastatin’ bound into the GPCR protein SAR0276 from MRSA252 with Ki=4.4 nmol

Photograph 8 shows the Statin Lovastatin bound into the Homo sapiens VDR with Ki=9.6 nmol.

Photograph 9 shows Simvastatin bound into the Homo Sapiens PPAR with Ki=0.3 nmol.

Photograph 10 shows the inter-atomic forces between the residues of the VDR and the atoms of 1,25-dihydroxyvitamin-D, the natural activator of the VDR (using ‘Ligplot’ standard software nomenclature)

Photograph 11 shows the inter-atomic forces between the residues of the VDR and the atoms of Olmesartan Medoxomil, a partial VDR Agonist. Note that there are strong interactions between Olmesartan and the residues Arg274, Tyr143, Ser275, SER278 and Ser237, which are essential for activation of the VDR.

Photograph 12 shows the position of the Vitamin D metabolites when docked into the VDR at the point of lowest energy. Note that, of all the D metabolites, only 1,25-D has the 1-alpha hydroxylation necessary to activate the receptor (note that Olmesartan Medoxomil has an oxygen in a symbiotic position, which is why it can act as an agonist).

Photograph 13 shows the inter-atomic forces between Olmesartan Medoxomil and the residues of a putative CB1 receptor, docked as a partial agonist, with affinity Ki=3 nanomolar.

### IN CONCLUSION

It will be clear that the invention may be practiced otherwise than as particularly described in the foregoing description and examples. Numerous modifications and variations of the present invention are possible in light of the above teachings and, therefore, are within the scope of the appended Claims.

[image]

[image]

[image]

[image]

[image]

[image]

[image]

[image]

[image]

[image]

[image]

[image]

[image]

TABLE 1
Estimated Inhibition Constant (Ki)(nanomolar) for ARBs and Statins binding
into VDR, PPAR (alpha&gamma) and GCR (smaller numbers, higher affinity)

| Estimated Inhibition Constant | | | | | | | |
|---|---|---|---|---|---|---|---|
| Ki, (nanomolar) | Candesartan | Irbesartan | Losartan | Olmesartan | Telmisartan | Valsartan | Atorvastatin |
| VDR | 30 | 10 | 74 | 10 | 0.04 | 14 | — |
| PPAR gamma | 61 | 6 | 3 | 12 | 0.3 | 12 | 4 |
| PPAR alpha | 3 | 0.9 | 4 | 3 | 0.7 | 26 | 2 |
| GCR | 6 | 0.8 | 4 | 1 | 2 | 10 | 1 |

| Estimated Inhibition Constant | | | | | |
|---|---|---|---|---|---|
| Ki, (nanomolar) | Fluvastatin | Lovastatin | Pravastatin | Rosuvastatin | Simvastatin |
| VDR | — | 10 | 62 | — | 4 |
| PPAR gamma | 12 | 0.2 | 21 | 24 | 0.3 |
| PPAR alpha | 1 | 19 | 2 | 18 | 4 |
| GCR | 3 | 15 | 8 | 7 | 2 |

Notes:
1. Low affinity Ki values (above 100 nanomolar) have been shown as a dash.
2. The Eprosartan daily dose is 600 mg, ten times that of the other drugs in the chart. Before comparison, Eprosartan's Ki values: 26, and 100; must be reduced by the dosage ratio. It was thus omitted from the table, to reduce confusion.

What is claimed is:

1. A method for treating and/or preventing Th1 immune conditions including Diabetes Type 1, Diabetes Type 2, Rheumatic Arthritis, Reactive Arthritis, Osteo Arthritis, Psoriasis, Scleroderma, Osteoporosis, Atherosclerosis, Myocarditis, Endocarditis, Pericarditis, Stroke, Embolism, Alzheimer's, Cystic Fibrosis, Hashimoto's Thyroiditis, Graves Disease, Leprosy, Syphilis, Lyme, Chronic Lyme, Borreliosis, Neuro-borreliosis, Inflammatory Bowel Disease (IBD), Tuberculosis, Latent Tuberculosis, Sarcoidosis, Neurosarcoidosis, Lupus, Discoid Lupus, Lupus Pernio, Lupus Nephritis, Systemic Lupus Erythematosis (SLE), Asthma, Macular Degeneration, Uveitis, Crohn's, Irritable Bowel Syndrome, Sjogren's, Fibromyalgia, Chronic Fatigue Syndrom (CFS), Chronic Fatigue Immune Dysfunction Syndrome (CFIDS), Myalgic Encephalitis (ME), Amyotrophic Lateral Sclerosis (ALS), Parkinson's, Multiple Sclerosis, Autism Spectrum Disorder (ASD), Attention Deficit Disorder (ADD), Attention Deficit Hyperactivity Disorder (ADHD), Schizophrenia, Obsessive Compulsive Disorder (OCD), Dysthymia, Bipolar Disorders, Epilepsy, Dementia, and Mania; comprising the administration of one or more chemical or biologic agents capable of displacing endogenous ligands from the Binding Pocket of the Receptors known as VDR, and/or PPAR, and/or GCR and/or CB1 and/or Angiotensin II Type 1, together with one or more chemical or biologic agents capable of inhibiting bacterial protein synthesis by inhibiting the actions of the 70S bacterial ribosome.

2. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan, administered in such a way that the concentration of ARB in the bloodstream is constrained from falling below 20% of its peak value by using a method of semi-continuous administration selected from a group consisting of trans-cutaneous-patch, implanted infusion device, implanted drug delivery system, external infusion device, trans-cutaneous delivery system, continuous Intravenous infusion.

3. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan, administered in such a way that the concentration of ARB in the bloodstream is constrained from falling below 20% of its peak value by using a method of intermittent administration selected from a group consisting of oral dosing at intervals sufficiently small to stabilize the ARB level between 20% and 100%, injections at intervals sufficiently small to stabilize the ARB level between 20% and 100% of its peak, intermittent Intravenous (IV) or trans-cutaneous infusion at interval sufficiently small to stabilize the ARB level between 20% and 100% of its peak value.

4. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan, administered using techniques designed to slow the release, or to slow the absorption, of the Angiotensin Receptor Blocker and thus stabilize its level at between 20% and 100%; such techniques include formulation with polymers or substances known as sustained-release binders, or other semi-soluble compounds.

5. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; and one or more of the antibiotics is selected from a group consisting of the 30S Bacterial Ribosomal subunit inhibitors Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin.

6. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; and one or more of the antibiotics is selected from a group consisting of the 30S Bacterial Ribosomal subunit inhibitors The Tetracycline family, or selected from a group consisting of the Tetracycline sub-type The Glycylcyclines.

7. The method defined in claim 6 wherein the 30S subunit inhibitor antibiotics are administered with a pulsatile dosing frequency between once every 36 hours and once every 10 days, such that the concentration of the antibiotic in plasma is allowed to drop before the next dose of antibiotic is administered.

8. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; and one or more of the antibiotics is selected from a group consisting of the 50S Bacterial Ribosomal subunit inhibitors Azithromycin, Clarithromycin, Chloramphenicol, Linezolid, Erythromycin, Roxithromycin, Troleandomycin, Tylocin, Carbomycin A, Clindamycin, Lincomycin, Cethromycin, Telithromycin, Sparsomycin, Tiamulin, Dalfopristin and Quinupristin.

9. The method defined in claim 8 wherein the 50S subunit inhibitor antibiotics are administered with a pulsatile dosing frequency between once every 36 hours and once every 45 days, such that the concentration of the antibiotic in plasma is allowed to drop before the next dose of antibiotic is administered.

10. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; and the 70S bacterial-ribosome is inhibited by two or more antibiotics selected so that both the 30S and 50S subunits are symbiotically inhibited from full bacterial protein synthesis.

11. The method defined in claim 10 wherein the 70S ribosome is inhibited by selecting one or more antibiotics from a group consisting of the 30S subunit inhibitors Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin; together with one or more antibiotics from a group consisting of the 50S subunit inhibitors Azithromycin, Clarithromycin, Clindamycin, Chloramphenicol, Linezolid, Erythromycin, Roxithromycin, Troleandomycin, Tylocin, Carbomycin A, Sparsomycin, Lincomycin, Cethromycin, Telithromycin, Tiamulin, Dalfopristin and Quinupristin.

12. The method defined in claim 11 wherein some, or all, of the antibiotics are administered with a pulsatile dosing frequency between once every 36 hours and once every 45 days, so that the concentration of the antibiotic in plasma is allowed to drop before the next dose of antibiotic is administered.

13. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; wherein the 70S bacterial-ribosome-inhibiting antibiotics comprise one or more selected from a group consisting of inhibitors of the 30S ribosomal sub-unit Minocycline, Minocycline hydrochloride, Demeclocycline and Demeclocycline hydrochloride; together with the 50S subunit inhibiting antibiotic Azithromycin; wherein the 30S subunit inhibitors are administered at a frequency between 36 and 8 days; and wherein the Azithromycin is administered at a frequency between 6 and 45 days.

14. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; and the 70S bacterial-ribosome-inhibiting antibiotics comprise one or more selected from a group consisting of inhibitors of the 30S ribosomal sub-unit Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin; together with one or more selected from a group consisting of the 50S subunit inhibiting antibiotics which bind near the PTC Clindamycin, Dalfopristin, Chloramphenicol, Linezolid, Tiamulin, and Lincomycin.

15. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; wherein the 70S bacterial-ribosome-inhibiting antibiotics comprise one or more antibiotics from a group consisting of the 30S subunit inhibitors Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin; together with one or more antibiotics from a group consisting of the 50S subunit inhibitors Azithromycin, Clarithromycin, Erythromycin, Roxithromycin, Troleandomycin, Tylocin, Carbomycin A, Sparsomycin, Lincomycin, Cethromycin, Telithromycin, and Quinupristin; together with one or more selected from a group consisting of the 50S subunit inhibiting antibiotics which bind near the PTC Clindamycin, Dalfopristin, Chloramphenicol, Linezolid, Tiamulin, and Lincomycin.

16. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; wherein the 70S bacterial-ribosome-inhibiting antibiotics comprise one or more antibiotics from a group consisting of the 30S subunit inhibitors Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin; together with two symbiotic 50S subunit inhibiting antibiotics Azithromycin and Clindamycin; wherein the 30S subunit inhibitors are administered at a frequency between 36 and 8 days; wherein the Azithromycin is administered at a frequency between 6 and 21 days; and wherein the Clindamycin is administered at a frequency between 36 hours and 21 days.

17. The method defined in claim 1 wherein one or more receptor binding agents are selected from a group consisting of the Sartan family of Angiotensin Receptor Blockers (ARB) Olmesartan, Olmesartan Medoxomil, Irbesartan, Eprosartan, Candesartan, Losartan, Telmisartan and Valsartan; wherein two or more 70S bacterial-ribosome-inhibiting antibiotics are selected, one from a group consisting of the 50S subunit inhibitors Azithromycin, Clarithromycin, Erythromycin, Roxithromycin, Troleandomycin, Tylocin, Carbomycin A, Sparsomycin, Lincomycin, Cethromycin, Telithromycin, and Quinupristin; together with one or more selected from a group consisting of the 50S subunit inhibiting antibiotics which bind near the PTC Clindamycin, Dalfopristin, Chloramphenicol, Linezolid, Tiamulin, and Lincomycin.

18. A method for treating and/or preventing Th1 immune conditions including Diabetes Type 1, Diabetes Type 2, Rheumatic Arthritis, Reactive Arthritis, Osteo Arthritis, Psoriasis, Scleroderma, Osteoporosis, Atherosclerosis, Myocarditis, Endocarditis, Pericarditis, Stroke, Embolism, Alzheimer's, Cystic Fibrosis, Hashimoto's Thyroiditis, Graves Disease, Leprosy, Syphilis, Lyme, Chronic Lyme, Borreliosis, Neuro-borreliosis, Inflammatory Bowel Disease (IBD), Tuberculosis, Latent Tuberculosis, Sarcoidosis, Neurosarcoidosis, Lupus, Discoid Lupus, Lupus Pernio, Lupus Nephritis, Systemic Lupus Erythematosis (SLE), Asthma, Macular Degeneration, Uveitis, Crohn's, Irritable Bowel Syndrome, Sjogren's, Fibromyalgia, Chronic Fatigue Syndrom (CFS), Chronic Fatigue Immune Dysfunction Syndrome (CFIDS), Myalgic Encephalitis (ME), Amyotrophic Lateral Sclerosis (ALS), Parkinson's, Multiple Sclerosis, Autism Spectrum Disorder (ASD), Attention Deficit Disorder (ADD), Attention Deficit Hyperactivity Disorder (ADHD), Schizophrenia, Obsessive Compulsive Disorder (OCD), Dysthymia, Bipolar Disorders, Epilepsy, Dementia, and Mania; comprising administration of two or more chemical or biologic agents capable of inhibiting bacterial protein synthesis by symbiotically inhibiting the actions of the 70S-bacterial-ribosome.

19. The method defined in claim 18 wherein one or more antibiotics are selected from a group consisting of 30S subunit inhibiting antibiotics Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin; and one or more antibiotic(s) are selected from either or both of the following symbiotic groups: a group consisting of 50S subunit-inhibiting-antibiotics Azithromycin, Clarithromycin, Erythromycin, Roxithromycin, Troleandomycin, Tylocin, Carbomycin A, Sparsomycin, Cethromycin, Telithromycin and Quinupristin; a group consisting of 50S subunit-inhibiting-antibiotics which bind near the PTC, Clindamycin, Dalfopristin, Chloramphenicol, Linezolid, Tiamulin, and Lincomycin.

20. The method described in claim 19 where the 30S subunit inhibiting antibiotics are administered with a pulsatile dosing frequency between once every 36 hours and once every 8 days, such that the concentration of the antibiotic in plasma is allowed to drop before the next dose of antibiotic is administered.

21. The method described in claim 19 where the period of treatment is 3 months or longer.

22. The method defined in claim 1 wherein one or more receptor binding agents are selected from a group consisting of the ‘Statin’ family of hypolipidemic agents Atorvastatin, Fluvastatin, Lovastatin, Pravastatin, Rosuvastatin and Simvastatin.

23. The method defined in claim 1 wherein one or more receptor binding agents are selected from a group consisting of the ‘Statin’ family of hypolipidemic agents Atorvastatin, Fluvastatin, Lovastatin, Pravastatin, Rosuvastatin and Simvastatin; and one or more of the antibiotics are selected from a group consisting of the 30S Bacterial Ribosomal subunit inhibitors Minocycline, Minocycline hydrochloride, Demeclocycline, Demeclocycline hydrochloride, Tigecycline, Tetracycline, Oxytetracycline, Doxycycline, Doxycycline hyclate, Spectinomycin, Hygromycin, Paromomycin, Streptomycin, Kanamycin, Gentamicin, Tobramycin, Amikacin, Netilmicin and Neomycin.

24. The method defined in claim 1 wherein one or more receptor binding agents are selected from a group consisting of the ‘Statin’ family of hypolipidemic agents Atorvastatin, Fluvastatin, Lovastatin, Pravastatin, Rosuvastatin and Simvastatin; and one or more of the antibiotics is selected from a group consisting of the 50S Bacterial Ribosomal subunit inhibitors Azithromycin, Clarithromycin, Chloramphenicol, Linezolid, Erythromycin, Roxithromycin, Troleandomycin, Tylocin, Carbomycin A, Clindamycin, Lincomycin, Cethromycin, Telithromycin, Sparsomycin, Tiamulin, Dalfopristin and Quinupristin.
